(19) United States
(12) Patent Application Publication
(10) Pub. No.: US 2011/0217371 A1
(43) Pub. Date: Sep. 8, 2011

(54) CONTROLLED-RELEASE MICROPARTICLES AND METHOD OF PREPARING SAME

(75) Inventors: Kwang-Hyun Shin, Gyeonggi-do (KR); Jaekwan Kim, Seoul (KR); Jai-ll Jun, Gyeonggi-do (KR); Deok-Ki Hong, Gyeonggi-do (KR); Joon-Ho Bae, Gyeonggi-do (KR)

(73) Assignee: AMOREPACIFIC CORPORATION, Seoul (KR)

(21) Appl. No.: 13/127,965

(22) PCT Filed: Nov. 10, 2009
(86) PCT No.: PCT/KR2009/006590

§ 371 Date: May 5, 2011

(30) Foreign Application Priority Data
Nov. 10, 2008 (KR) 10-2008-0111234

(51) Int. Cl. A61K 9/00; A61K 9/24; A61K 9/52; A61K 31/18; A61P 3/10; A61P 11/08; A61P 11/14; A61P 9/06; A61P 23/02; A61P 25/08; A61P 1/04; A61P 25/00; A61P 3/06; A61P 11/06; A61P 13/08; A61P 25/24; A61P 17/02; A61P 9/12; A61P 9/04

(52) U.S. Cl. 424/457; 424/484; 424/472; 514/603; 424/486

(57) ABSTRACT

Disclosed is a controlled-release microparticle: including a matrix comprising a pharmacologically active component; and a controlled-release layer comprising a substance which forms a controlled-release stratum on the matrix. The disclosed controlled-release microparticle not only allows effective dual release control of a drug but can also exhibit outstanding dissolution characteristics even when a small amount of coating substance is used.

### TECHNICAL FIELD

The present disclosure relates to a controlled-release microparticle including a matrix comprising a pharmacologically active component, and a method for preparing the same.

### BACKGROUND ART

Many pharmacologically active substances including drugs and prodrugs are prepared into orally administrable preparations allowing controlled release (also known as slow release or sustained release).

When such a drug as tamsulosin hydrochloride is administered in the form of a controlled-release tablet, increase of blood level and concentration-dependent side effects resulting therefrom occur frequently because of its nonuniform rate of passing through the pyloric sphincter. Thus, there have been attempts to prepare a tablet with a small particle diameter that is disintegrated quickly in the buccal cavity.

For instance, Korean Patent No. 0530546 discloses a composition for a tablet comprising drug-containing controlled-release microparticles of 350 μm or smaller, an excipient and a binder. However, the patent relates to controlled-release microparticles without a matrix comprising a drug. The disclosed controlled-release microparticles are disadvantageous in that control of drug release is difficult, a large amount of coating substance is required to achieve controlled release of the microparticles, and a long coating time is required.

### DISCLOSURE

### Technical Problem

The present disclosure is directed to providing a controlled-release microparticle allowing easy control of drug release.

The present disclosure is also directed to providing a method for preparing a controlled-release microparticle allowing easy control of drug release.

### Technical Solution

In one general aspect, the present disclosure provides a controlled-release microparticle including: a matrix comprising a pharmacologically active component; and a controlled-release layer comprising a substance which forms a controlled-release stratum on the matrix. The pharmacologically active component may be uniformly dispersed or distributed in the matrix.

In another general aspect, the present disclosure provides a method for preparing a controlled-release microparticle, including: preparing a matrix comprising a pharmacologically active component; and forming a controlled-release layer comprising a substance which forms a controlled-release stratum on the matrix.

### ADVANTAGEOUS EFFECTS

The controlled-release microparticle of the present disclosure not only allows effective release control of a drug but can also exhibit outstanding dissolution characteristics even when a small amount of coating substance is used.

### DESCRIPTION OF DRAWINGS

FIG. 1 shows a dissolution test result of controlled-release microparticles prepared in Example 5 and Comparative Example 1.

### MODE FOR INVENTION

The present disclosure provides a dual controlled-release system comprising: a matrix comprising a pharmacologically active component; and a controlled-release layer comprising a substance which forms a controlled-release stratum on the matrix. The matrix allows primary controlled release of the active component, and the additional coating layer allows secondary controlled release of the active component. The amount of the coating substance and coating time can be reduced remarkably. Consequently, comparable or better dissolution characteristics can be exhibited even when a smaller amount of coating substance is used.

In an embodiment, the substance which forms a controlled-release stratum may be a polymer selected from a group consisting of a water-insoluble polymer, a gastric-soluble polymer, an enteric-soluble polymer, a water-soluble polymer, and a mixture thereof. The polymer material may be selected adequately depending on purposes.

The substance which forms a controlled-release stratum may be a pH-independent water-insoluble polymer for enabling controlled drug release. For example, the water-insoluble polymer may be one or more selected from a group consisting of a ethyl cellulose, water-insoluble cellulose ether such as Aquacoat (FMC), etc., an ethyl acrylate-methyl methacrylate-chlorotrimethylammoniumethyl methacrylate copolymer (e.g., Eudragit RS, Evonik), polyvinyl acetate, an ethyl acrylate-methyl methacrylate copolymer, and a dispersion thereof (As an example of the ethyl acrylate-methyl methacrylate copolymer dispersion, Eudragit NE30D (Evonik) may be used.).

For example, the gastric-soluble polymer may be one or more selected from a group consisting of polyvinyl acetal diethylamino acetate, and a methyl methacrylate-butyl methacrylate-dimethylaminoethyl methacrylate copolymer (e.g., Eudragit E, Evonik).

The substance which forms a controlled-release stratum may be an enteric-soluble polymer for providing enteric solubility. For example, the enteric-soluble polymer may be one or more selected from a group consisting of hydroxypropyl methylcellulose acetate succinate, hydroxypropyl methylcellulose phthalate, hydroxymethylethyl cellulose phthalate, carboxymethylethyl cellulose, a methacrylic acid-methyl methacrylate copolymer (e.g., Eudragit L100 and Eudragit S, Evonik), and a methacrylic acid-ethyl acrylate copolymer (e.g., Eudragit L100-55 and Eudragit L30D55, Evonik).

For example, the water-soluble polymer may be one or more selected from a group consisting of hydroxypropyl cellulose, hydroxypropyl methylcellulose, polyvinylpyrrolidone, and polyvinyl alcohol.

These polymer materials may be used alone or in combination to attain desired controlled release.

In an embodiment, the controlled-release layer may account for 15-60 wt %, more specifically 20-40 wt %, based on the total weight of the controlled-release microparticle. Within the aforesaid range, drug release may be effectively controlled and coating may be accomplished in short time.

The active component may be uniformly dispersed or distributed in the matrix. The active component may be any therapeutically or prophylactically active component requiring controlled release, without particular limitation.

For example, the active component may be selected from a group consisting of: an antidiabetic selected from a group consisting of acetohexamide, insulin, tolbutamide, desmopressin, and glipizide; a diuretic selected from a group consisting of hydrochlorothiazide, polythiazide, and triamterene; a bronchodilator selected from a group consisting of aminopyrine, formoterol maleate, and theophylline; an antitussive selected from a group consisting of codeine phosphate, noscapine, dimemorfan phosphate, and dextromethorphan; an antiarrhythmic agent selected from a group consisting of quinidine nitrate, digitoxin, propafenone hydrochloride, and procainamide; a topical anesthetic selected from a group consisting of ethyl aminobenzoate, lidocaine, and dibucaine hydrochloride; an antiepileptic selected from a group consisting of phenytoin, ethosuximide, and primidone; a synthetic adrenocortical steroid selected from a group consisting of hydrocortisone, prednisolone, triamcinolone, and betamethasone; a peptic ulcer drug selected from a group consisting of famotidine, ranitidine hydrochloride, cimetidine, sucralfate, sulpiride, teprenone, plaunotol, 5-aminosalicylic acid, sulfasalazine, omeprazole, pantoprazole, and lansoprazole; a central nervous system drug selected from a group consisting of indeloxazine, idebenone, tiapride hydrochloride, bifemelane hydrochloride, and calcium homopantothenate; an antihyperlipidemic agent selected from a group consisting of pravastatin sodium, simvastatin, lovastatin, fluvastatin, and atorvastatin; an antibiotic selected from a group consisting of phthalylampicillin hydrochloride, cefotetan, and josamycin; a benign prostatic hypertrophy therapeutic agent selected from a group consisting of tamsulosin hydrochloride, doxazosin mesylate, and terazosin hydrochloride; an antiasthmatic agent selected from a group consisting of pranlukast, the philcast, albuterol, ambroxol, budesonide, and levalbuterol; a gastroprokinetic agent selected from a group consisting of mosapride, mosapride citrate, itopride, itopride hydrochloride, cisapride, cisapride monohydrate, cisapride tartrate, domperidone, domperidone maleate, metoclopramide, metoclopramide hydrochloride, trimebutine, trimebutine maleate, clebopride, clebopride maleate, bromopride, and levosulpiride; an anti-depressant; a peripheral circulation improving agent; an antithrombotic agent; an antihypertensive; a heart failure drug; a diabetic complication drug; a skin ulcer drug; and a combination thereof.

In the matrix, the active component may exist alone or in combination of two or more. The active component may be present in any therapeutically effective amount without particular restriction. For example, the active component may be present in the matrix in an amount of 1-80 wt %, specifically 5-30 wt %, based on the total weight of the matrix.

In a specific embodiment, the matrix may comprise an excipient and a binder.

The excipient included in the matrix is not particularly restricted as long as it has properties appropriate for the formation of the matrix, and may be selected adequately depending on purposes. For example, the excipient may be selected from a group consisting of an organic excipient such as a cellulose derivative and a saccharide, an inorganic excipient such as calcium phosphate, and a mixture thereof. The cellulose derivative may be selected from a group consisting of microcrystalline cellulose and low-substituted hydroxypropyl cellulose, the saccharide may be selected from a group consisting of lactose, starch and pregelatinized starch, and the calcium phosphate may be one or more selected from a group consisting of anhydrous calcium hydrogen phosphate, calcium hydrogen phosphate dihydrate, and tricalcium phosphate.

The amount of the excipient in the matrix may be controlled adequately depending on the dose of the drug and/or the final size of the microparticle. It may be present in an amount of 20-99 wt %, specifically 70-95 wt %, based on the matrix. Within the aforesaid range, drug release can be controlled effectively only with the matrix.

The binder included in the matrix is not particularly restricted as long as it acts as a binder for preparation of the microparticle. For example, the binder may be one or more selected from water, an aqueous suspension of a methacrylic acid copolymer, an aqueous suspension of ethyl cellulose, and an aqueous suspension of polyvinyl acetate.

The amount of the binder in the matrix may be controlled adequately depending on the dose of the drug and/or the final size of the microparticle. It may be present in the matrix in an amount of more than 0 but below 30 wt %, specifically more than 0 but below 10 wt %, based on solid content.

In an embodiment, the controlled-release microparticle may have an average particle diameter of, for example, 300 μm or smaller, 250 μm or smaller, or 200 μm or smaller. Furthermore, the controlled-release microparticle may have an average particle diameter of, for example, 300-100 μm, 300-150 μm, 250-100 μm, 250-150 μm, or 200-100 μm. In general, a large amount of coating substance and a long coating time are required to enable controlled release of a microparticle which is 300 μm or smaller. However, the controlled-release microparticle of the present disclosure, which comprises the matrix comprising the pharmacologically active component and the controlled-release layer comprising the substance which forms a controlled-release stratum on the matrix, allows effective control of release even when the particle size is 300 μm or smaller and exhibits comparable or better dissolution characteristics even when a smaller amount of coating substance is used.

As occasion demands, the controlled-release microparticle may be prepared into a tablet such as a quickly disintegrating tablet, a suspension tablet or a chewable tablet or into a capsule by a common tablet making method or by humidifying/drying or heating as required. For this purpose, a pharmaceutically acceptable additive may be further included, common examples of which include a plasticizer, a lubricant, and other supplementary aids.

The present disclosure also provides a method for preparing a controlled-release microparticle, comprising: preparing a matrix comprising a pharmacologically active component; and forming a controlled-release layer comprising a substance which forms a controlled-release stratum on the matrix.

In an embodiment, in the step of preparing the matrix comprising a pharmacologically active component, a drug, an excipient and a binder may be mixed until the mixture becomes homogeneous to prepare the matrix. The excipient and the binder are the same as those described above.

An apparatus used to prepare the matrix is not particularly restricted. For example, such apparatus as a flow type granulator or a high shear mixer may be used. The diameter of the matrix may be controlled adequately such that the final diameter of the controlled-release microparticle may be 300 μm or smaller.

Through this, the drug may be uniformly distributed in the matrix, and the drug and the excipient may be bound together by the binder. As a result, the matrix can release the drug as desired through diffusion and erosion.

Next, the controlled-release layer comprising the substance which forms a controlled-release stratum is formed on the matrix. The substance which forms a controlled-release stratum may be different polymer layers depending on purposes. For example, the polymer layer may be formed by spraying a solution in which the polymer component is dissolved to the matrix to a thickness required to attain the desired drug release rate. The polymer that may be used for the polymer layer is the same as described above.

The examples and experiments will now be described. The following examples and experiments are for illustrative purposes only and not intended to limit the scope of the present disclosure.

<Preparation of Matrix>

### Example 1

Tamsulosin hydrochloride (3.33 g) was adequately triturated and mixed with microcrystalline cellulose powder (Vivapur PH101, 496.67 g). Then, a spherical matrix comprising tamsulosin hydrochloride was prepared while spraying water (500 g) using a rotary-type fluidized bed apparatus (GPCG-1, Glatt, Germany).

Among the prepared particles, only those having a particle diameter of 150-250 μm (60-100 mesh) were selected.

### Example 2

A spherical matrix was prepared in the same manner as in Example 1, except for spraying a dispersion comprising Eudragit L30D-55 (88.90 g; solid 26.67 g (solid content=30%), water 62.23 g) and water (437.77 g). Only the particles having a particle diameter of 150-250 μm (60-100 mesh) were selected.

### Example 3

Tamsulosin hydrochloride (3.33 g) was adequately triturated and mixed with microcrystalline cellulose (346.67 g), calcium hydrogen phosphate (100 g) and lactose (50 g). Then, a spherical matrix was prepared while spraying a dispersion comprising Eudragit L30D-55 (88.90 g; solid 26.67 g (solid content=30%), water 62.23 g) and water (437.77 g).

Among the prepared particles, only those having a particle diameter of 150-250 μm (60-100 mesh) were selected.

<Preparation of Controlled-Release Microparticles>

### Example 4

To the tamsulosin hydrochloride-containing matrix (800 g) prepared in Example 1, a 30% aqueous dispersion of ethyl cellulose (ECD, 206.3 g; solid 61.89 g) and a mixed aqueous dispersion (533.33 g, solid content 15%) of Kollicoat IR (3.26 g, weight ratio=9.5:0.5) and triethyl citrate (14.85 g) were repeatedly sprayed by bottom spraying using the same fluidized bed apparatus to coat to a weight ratio of 10%, 20%, 30% and 40% based on the weight of the microparticle. Then, by hardening at room temperature and 60° C. for 12 hours, respectively, controlled-release microparticles with an average particle diameter of 250 μm were prepared.

### Example 5

To the tamsulosin hydrochloride-containing matrix (800 g) prepared in Example 1, an ECD (190.5 g, solid 57.15 g) and a mixed aqueous dispersion (533.33 g, solid content 15%) of Kollicoat IR (6.35 g, weight ratio=9:1) and triethyl citrate (13.70 g) were repeatedly sprayed to coat to a weight ratio of 10%, 15%, 20% and 30% based on the weight of the microparticle. Then, by hardening at room temperature and 60° C. for 12 hours, respectively, controlled-release microparticles with an average particle diameter of 230 μm were prepared.

Each controlled-release microparticle (800 g) coated with a weight ratio of 15%, 20% and 30%, respectively, was coated by bottom spraying using the same fluidized bed apparatus with a mixed solution of Eudragit L30D-55 (381.3 g, solid 114.3 g), talc (34.2 g), triethyl citrate (11.4 g) and purified water (373 g) to a weight ratio of 20% based on the weight of the controlled-release microparticles. Thus, enteric-soluble controlled-release microparticles with an average particle diameter of about 250 μm, coated with a weight ratio of 35%, 40% and 50% were prepared.

### Example 6

Controlled-release microparticles with an average particle diameter of 230 μm were prepared in the same manner as in Example 4, except for spraying an ECD (680 g, solid 204 g) and a mixed aqueous dispersion (1984 g, solid content=15.0%) of Kollicoat IR (36 g, weight ratio=8.5:1.5) and triethyl citrate (57.6 g).

### Example 7

To the tamsulosin hydrochloride-containing matrix (800 g) prepared in Example 2, an ECD (680 g, solid 204 g) and a mixed aqueous dispersion (1984 g, solid content 15.0%) of Kollicoat IR (36 g, weight ratio=8.5:1.5) and triethyl citrate (57.6 g) were repeatedly sprayed to coat to a weight ratio of 37.2% based on the weight of the microparticle. Then, by hardening at 60° C. for 12 hours, a controlled-release microparticle with an average particle diameter of 250 μm was prepared.

### Comparative Example 1

Tamsulosin hydrochloride (20 g) and hydroxypropyl methylcellulose (20 g) were dissolved in a mixed solution of purified water (76 g) and methanol (684 g). After putting an inert core (spherical microcrystalline cellulose particle, 1000 g) having a particle diameter of approximately 50-150 μm in a rotary-type fluidized bed apparatus (GPCG-1, Glatt, Germany), the core was coated with the mixed solution to prepare a tamsulosin hydrochloride microparticle.

Separately from this, ethyl cellulose (133.25 g) and hydroxypropyl methylcellulose (46.75 g) were dissolved in a mixed solution of purified water (174.5 g) and methanol (5645.5 g) to prepare a coating solution.

The tamsulosin hydrochloride microparticle (1000 g) was put in the same fluidized bed apparatus and coated with the separately prepared coating solution to a weight ratio of 18% based on the microparticle to prepare a controlled-release microparticle.

The prepared controlled-release microparticle (1000 g) was put in the same fluidized bed apparatus and coated with a mixed solution of an ECD (500 g), Eudragit L30D-55 (1000 g), Eudragit NE30D (166.75 g) and purified water (1666.75 g) to a weight ratio of 50% based on the microparticle to prepare an enteric-soluble controlled-release microparticle with an average diameter of about 250 μm.

<Test Example: Dissolution Test>

Each of the tamsulosin hydrochloride 0.2 mg microparticles prepared in Examples and Comparative Example 1 was filled in a capsule and dissolution rate was compared according to the Korean Pharmacopeia Dissolution Test No. 2. The test was performed at 75 rpm, using a disintegration test solution no. 2 (pH 6.8, 500 mL). Samples (10 mL) were taken at 30 minutes, 1 hour and 4 hours after initiation of the dissolution test. After addition of 0.5 N HCl (1.0 mL) followed by filtration, the filtrate was quantitated by high-performance liquid chromatography (HPLC) under the following conditions. 6 samples were tested for each microparticle.

Column: LUNA C18 (4.6×150 mm, 5 μm)

Detector: UV 225 nm

Flow rate: to maintain tamsulosin residence time at about 6 minutes

Sample injection volume: 100 μL

Column temperature: 40° C.

Mobile phase: Perchloric acid (8.7 mL) and sodium hydroxide (3.0 g) dissolved in water (1900 mL) to prepare a sodium hydroxide test solution. After adjusting to pH 2.0, water was added to make final volume 2000 mL. Acetonitrile (600 mL) was added to the resultant solution (1400 mL) for use as the mobile phase.

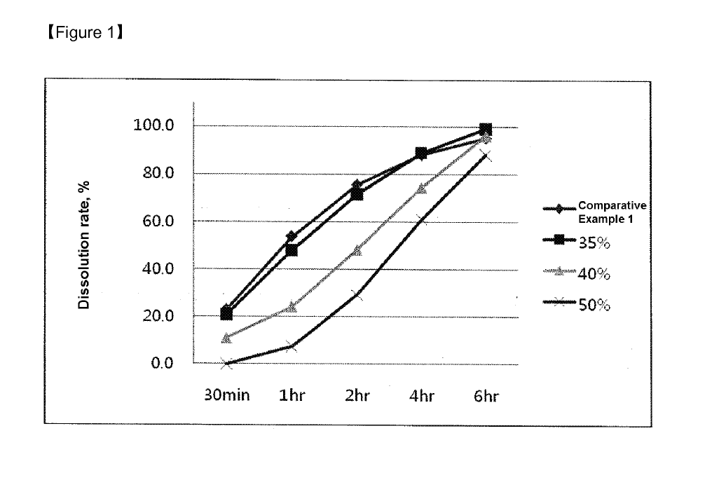

The result is shown in FIG. 1. It can be seen that the controlled-release microparticle of Example 5 (coated to a weight ratio of 35%) exhibits a similar dissolution profile as that of Comparative Example 1 (coated to a weight ratio of 58%). Consequently, it can be confirmed that the controlled-release microparticle according to the present disclosure exhibits comparable dissolution characteristics as Comparative Example 1 even with a smaller amount of coating substance and less coating time.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A controlled-release microparticle comprising:
a matrix comprising a pharmacologically active component; and
a controlled-release layer comprising a substance which forms a controlled-release stratum on the matrix.

2. The controlled-release microparticle according to claim 1, wherein the substance which forms a controlled-release stratum is a polymer selected from a group consisting of a water-insoluble polymer, a gastric-soluble polymer, an enteric-soluble polymer, a water-soluble polymer, and a mixture thereof.

3. The controlled-release microparticle according to claim 2, wherein the water-insoluble polymer is one or more selected from a group consisting of ethyl cellulose, cellulose ether, an ethyl acrylate-methyl methacrylate-chlorotrimethylammoniumethyl methacrylate copolymer, polyvinyl acetate, an ethyl acrylate-methyl methacrylate copolymer, and a dispersion thereof.

4. The controlled-release microparticle according to claim 2, wherein the gastric-soluble polymer is one or more selected from a group consisting of polyvinyl acetal diethylamino acetate, and a methyl methacrylate-butyl methacrylate-dimethylaminoethyl methacrylate copolymer.

5. The controlled-release microparticle according to claim 2, wherein the enteric-soluble polymer is one or more selected from a group consisting of hydroxypropyl methylcellulose acetate succinate, hydroxypropyl methylcellulose phthalate, hydroxymethylethyl cellulose phthalate, carboxymethylethyl cellulose, a methacrylic acid-methyl methacrylate copolymer, and a methacrylic acid-ethyl acrylate copolymer.

6. The controlled-release microparticle according to claim 2, wherein the water-soluble polymer is one or more selected from a group consisting of hydroxypropyl cellulose, hydroxypropyl methylcellulose, polyvinylpyrrolidone, and polyvinyl alcohol.

7. The controlled-release microparticle according to claim 1, wherein the controlled-release layer accounts for 15-60 wt % based on the total weight of the controlled-release microparticle.

8. The controlled-release microparticle according to claim 1, wherein the active component is selected from a group consisting of: an antidiabetic selected from a group consisting of acetohexamide, insulin, tolbutamide, desmopressin, and glipizide; a diuretic selected from a group consisting of hydrochlorothiazide, polythiazide, and triamterene; a bronchodilator selected from a group consisting of aminopyrine, formoterol maleate, and theophylline; an antitussive selected from a group consisting of codeine phosphate, noscapine, dimemorfan phosphate, and dextromethorphan; an antiarrhythmic agent selected from a group consisting of quinidine nitrate, digitoxin, propafenone hydrochloride, and procainamide; a topical anesthetic selected from a group consisting of ethyl aminobenzoate, lidocaine, and dibucaine hydrochloride; an antiepileptic selected from a group consisting of phenytoin, ethosuximide, and primidone; a synthetic adrenocortical steroid selected from a group consisting of hydrocortisone, prednisolone, triamcinolone, and betamethasone; a peptic ulcer drug selected from a group consisting of famotidine, ranitidine hydrochloride, cimetidine, sucralfate, sulpiride, teprenone, plaunotol, 5-aminosalicylic acid, sulfasalazine, omeprazole, pantoprazole, and lansoprazole; a central nervous system drug selected from a group consisting of indeloxazine, idebenone, tiapride hydrochloride, bifemelane hydrochloride, and calcium homopantothenate; an antihyperlipidemic agent selected from a group consisting of pravastatin sodium, simvastatin, lovastatin, fluvastatin, and atorvastatin; an antibiotic selected from a group consisting of phthalyl ampicillin hydrochloride, cefotetan, and josamycin; a benign prostatic hypertrophy therapeutic agent selected from a group consisting of tamsulosin hydrochloride, doxazosin mesylate, and terazosin hydrochloride; an antiasthmatic agent selected from a group consisting of pranlukast, the philcast, albuterol, ambroxol, budesonide, and levalbuterol; a gastroprokinetic agent selected from a group consisting of mosapride, mosapride citrate, itopride, itopride hydrochloride, cisapride, cisapride monohydrate, cisapride tartrate, domperidone, domperidone maleate, metoclopramide, metoclopramide hydrochloride, trimebutine, trimebutine maleate, clebopride, clebopride maleate, bromopride, and levosulpiride; an anti-depressant; a peripheral circulation improving agent; an antithrombotic agent; an antihypertensive; a heart failure drug; a diabetic complication drug; a skin ulcer drug; and a combination thereof.

9. The controlled-release microparticle according to claim 1, wherein the matrix further comprises an excipient and a binder.

10. The controlled-release microparticle according to claim 9, wherein the excipient is selected from a cellulose derivative, a saccharide, calcium phosphate, and a mixture thereof.

11. The controlled-release microparticle according to claim 10, wherein the cellulose derivative is selected from a group consisting of microcrystalline cellulose and low-substituted hydroxypropyl cellulose, the saccharide is selected from a group consisting of lactose, starch and pregelatinized starch, and the calcium phosphate is selected from a group consisting of anhydrous calcium hydrogen phosphate, calcium hydrogen phosphate dihydrate, and tricalcium phosphate.

12. The controlled-release microparticle according to claim 9, wherein the binder is one or more selected from water, an aqueous suspension of a methacrylic acid copolymer, an aqueous suspension of ethyl cellulose, and an aqueous suspension of polyvinyl acetate.

13. The controlled-release microparticle according to claim 1, wherein the controlled-release microparticle has an average particle diameter of 300 μm or smaller.

14. The controlled-release microparticle according to claim 1, wherein the controlled-release microparticles is prepared into a tablet or a capsule.

15. A method for preparing the controlled-release microparticle according to claim 1, comprising:
preparing a matrix comprising a pharmacologically active component; and
forming a controlled-release layer comprising a substance which forms a controlled-release stratum on the matrix.
